(19) United States
(12) Patent Application Publication
(10) Pub. No.: US 2021/0068987 A1
(43) Pub. Date: Mar. 11, 2021

(54) METHOD FOR PRODUCING AN ORTHOPEDIC DEVICE

(71) Applicant: Ottobock SE & Co. KGaA, Duderstadt (DE)

(72) Inventors: Mario KOPPE, Göttingen (DE); Jonas BORNMANN, Duderstadt (DE); Andreas LEINIGER, Leinefeld (DE)

(73) Assignee: Ottobock SE & Co. KGaA, Duderstadt (DE)

(21) Appl. No.: 17/054,635

(22) PCT Filed: May 9, 2019
(86) PCT No.: PCT/EP2019/061988

§ 371 Date: Nov. 11, 2020

(30) Foreign Application Priority Data
May 14, 2018 (DE) 10 2018 111 442.7

(51) Int. Cl. A61F 2/50; A61F 5/01; B33Y 80/00

(52) U.S. Cl. CPC A61F 2/5046; A61F 2/80; B33Y 80/00; A61F 5/0102

(57) ABSTRACT

A method for producing an orthopedic device, wherein the method comprises: providing a main body of the orthopedic device that comprises at least one volume that can be filled with a fluid, at least one supply line to this volume, and a flexible and/or elastic material in at least some sections of the main body in which there is no volume that can be filled with a fluid, arranging the main body on a body part of a patient, at least partially filling the volume with a curable fluid, and curing the curable fluid.

The invention relates to a method for producing an orthopedic device.

In the present case, an orthopedic device is understood particularly to mean orthoses and prostheses that can be produced in a wide range of ways. In many cases, they have to be individually adjusted to fit the body shape of the respective body part of the wearer or patient. In the case of prostheses, this applies especially to the prosthesis socket which, for instance in the case of a leg prosthesis, accommodates the amputation stump. To render it as comfortable to wear as possible, even when subjected to high loads, the prosthesis socket must be individually adjusted to fit the shape of the amputation stump. However, this not only applies to lower limb prostheses, but also, for example, to arm or hand prostheses. It is also often necessary to adapt orthoses, such as splint systems, to the individual physical conditions of the wearer or patient. In particular, orthoses must follow malpositions of joints or body parts, such that different components of an orthosis, such as splints and/or joints of a splint system, must be adapted to each other on an individual basis.

Traditionally, the production of a prosthesis socket for a leg prosthesis, for instance, comprises the execution of several process steps. Conventionally, an impression is first made of the amputation stump. This is used to produce a positive model of the stump. Different methods for producing this model and the impression are known from the prior art. However, it is disadvantageous that, regardless of the method used to create the model and the impression, different process steps are required.

This is often followed by the modelling of a trial socket, but preferably an end socket, on the positive model of the amputation stump, wherein said socket is subsequently adapted to fit the patient. At this point, minor changes, such as padding or the removal of material, can still be undertaken. Small errors can thus be rectified. In the case of major errors, such as an incorrect positioning of adapter elements via which other prosthesis components, such as a knee, lower leg or prosthetic feet, can be arranged on the socket, a new prosthesis socket must be produced and the method carried out again in full.

The prior art comprises various proposals for simplifying the production of a prosthesis socket. DE 10 2016 201 002 A1 describes a method in which 3D data taken from the amputation stump by a scanner is fed to a 3D printer. This printer prints a framework of the socket, which is subsequently cast.

EP 1 854 621 B1 describes a method in which a placeholder material is initially used to create a shape of the socket, which is subsequently impregnated with polyurethane, thus achieving the actual layer structure of the socket.

U.S. Pat. No. 7,438,843 B2 proposes constructing the prosthesis socket directly on the amputation stump in several steps. Errors in the production of the sockets and in particular in the mould to be cast cannot be corrected in any of these proposals. U.S. Pat. No. 6,991,444 B1 contains a similar approach. It also describes working directly on the stump of the patient. FR 2095097 comprises a method in which an amputation stump fitted with a liner is inserted into a volume that is subsequently filled with a curable compound.

EP 1 854 621 B1 describes that a PU layer is initially applied and then covered with a reinforcing layer, which is again covered with a PU layer. In this case, the PU layers are cast with uncured PU after they have been arranged, so that the two layers come into contact with one another through the reinforcing layer and bond as the PU cures.

DE 10 2014 219 570 B4 describes a method in which a volume that is to be filled with a matrix material is evacuated by covering it with a layer that is permeable to gas, to whose outer side a negative pressure is applied.

WO 2018/054966 A1 describes a device that features a prosthetic shoe made of a flexible material. This prosthetic shoe is formed of a skin contact surface and a socket sleeve layer which are joined together at the edge, for example sewn together. The resulting gap can be filled with foam when the prosthetic shoe is positioned on the amputation stump, thereby ensuring an optimum fit. To ensure sufficient stability, the prosthetic shoe formed in this way must be arranged in a separate outer shell, which must be tensioned by means of clamping devices.

The invention thus aims to propose a method for producing an orthopedic device with which the method can be carried out more quickly and therefore more cost-effectively.

The invention solves the problem by means of a method for producing an orthopedic device, wherein the method comprises the following steps:
a) providing a main body of the orthopedic device that comprises at least one volume that can be filled with a fluid and at least one supply line to said volume and a flexible and/or elastic material in at least some sections, in which there is no volume that can be filled with a fluid.
b) arranging the main body on a body part of the patient,
c) at least partially filling the volume with a curable fluid, and
d) curing the curable fluid.

Preferably, the entire main body, which is made of a main body material, is flexible and/or elastic. It can therefore be adapted to fit the shape of the body part. The main body has a volume provided, for example, in the form of ducts or tubes. It may also comprise at least one or several chambers. This main body is moulded on the patient's body part, for instance an amputation stump. To this end, a prosthetic liner may initially be pulled over the amputation stump, so that the shape of the moulded main body corresponds to the shape of the prosthesis socket required. Due to the flexibility of the main body material, the main body is moulded to the body part. The same applies if the orthopedic device to be produced is not a prosthesis socket, but rather a different prosthesis component or an orthosis. Irrespective of the type of orthopedic device, it is advantageous to arrange the main body on the patient's body in the same way as the orthopedic device to be produced.

At least one section, but preferably several sections, of the main body comprises a flexible and/or elastic material in which there is no volume that can be filled with a fluid. Therefore, this at least one section does not increase in stiffness upon the curing of the fluid; rather, it retains its flexible and/or elastic properties. This renders it possible to make the orthopedic device more comfortable to wear. This applies particularly if this at least one section comes into contact with an especially sensitive region of the amputation stump while the orthopedic device is being worn.

The volume is subsequently at least partially filled with a curable fluid. To this end, the volume has a supply line. The fluid can be introduced into the volume via a supply tube that can be connected to the supply line. The fluid spreads in the volume and at least partially fills it. If the volume is completely filled, this ensures that the pressure within the volume is constant and that more fluid can thus be filled into the respective volume at, for example, points on the main body that rest on recesses of the amputation stump than at other points. This results in an individual moulding of the main body to the body part of the patient.

The properties of the fluid can be adapted to fulfil the respective purpose. Preferably a curable 2 component fluid, such as a 2 K-Epoxy, is used. Fillers such as minerals or fibers can be used to lend the fluid the desired mechanical properties. This may also be done on a section-by-section basis, for example to mechanically strengthen regions of the main body that are subjected to especially high levels of strain. Of course, the fluid can also be used completely with fillers. Polyurethane foams can also be used in various compositions.

Following the at least partial filling of the volume with the curable fluid, the fluid is cured. In the case of a fluid that can be cured using radiation, for instance, this may be achieved by supplying energy in the form of electron beams or UV rays or, in the case of other fluid types, through heating or waiting. It has been proven advantageous if the fluid can be cured when the main body is positioned on the body part of the patient. This may be done, for example, via radiation with UV rays. This ensures that the curable fluid cures in exactly the position that corresponds to the optimum shape of the orthopedic device to be produced.

It is advantageous, however, if the curable fluid not only serves to optimally adapt the main body and thus the orthopedic device to the physical conditions of the patient, but if the volume also has at least a partial volume which, after the filled fluid has cured, fulfils a supporting function for the orthopedic device. In this way, for example, in the case of an upper leg prosthesis socket, the proximal edge, the distal end where, for instance, an adapter element is arranged, and/or at least one support between these two regions can be formed by a volume filled with the curable fluid. The main body with the filled volume can thus already serve as a final orthopedic device. It is therefore not necessary to first use the method described to produce a preliminary device, i.e. only a temporary device, the dimensions and model of which are used to mould the final orthopedic device. Rather, a method according to the invention can be used to produce the final orthopedic device.

Preferably, the fillable volume is situated at least partially, but preferably completely, in the flexible and/or elastic region of the main body.

It is especially preferable if the cured fluid forms bar-shaped or finger-shaped parts, wherein a section of the flexible and/or elastic material is situated between at least two of these parts. This enables an optimum combination of flexible and/or elastic sections, which are important in terms of comfort, and sections that can be and are later filled with fluid, which are required to ensure sufficient stability.

It is advantageous for the main body to be moulded to the patient's body part after the volume has been at least partially filled with the fluid. This allows the fluid to be pressed or pushed into different regions of the volume to achieve an optimum adaptation. Alternatively or additionally, the fluid can also be removed or at least partially removed from certain regions of the volume in order to achieve a lighter design or to reduce the pressure of the fluid in these regions of the volume.

In a preferred embodiment, the fluid is already present in one part of the volume. To fill the fillable volume, a container that holds the fluid, for instance, must be destroyed, for example bent or broken, thereby releasing the fluid in the fillable volume. Preferably, the fluid is a 2 component fluid, wherein the components are provided separately and are only released and come into contact with one another due to the destruction of at least one of the containers.

The main body preferably features several components, each of which comprises at least one partial volume that can be filled with the fluid. This enables different components to be joined together in the manner of a construction set in order to produce the main body in the desired form. This is especially, but not exclusively, advantageous for the production of orthoses. An orthosis which extends, for instance, along a majority of a limb, such as a leg or an arm, features different components that have to be connected to one another. This may refer, for example, to fixing elements for fixing the orthosis to the body part of the patient, splint elements and joints, which have to be connected to one another and positioned on an individual basis relative to one another in order to follow the individual physical conditions of the patient. In the preferred embodiment of the method, these components are provided as separate components that can be connected to each other and which each have at least one volume that can be filled with the curable fluid.

Preferably, upon connecting these several elements, at least two, but preferably several or even all partial volumes of the individual components, are connected to one another, thereby forming a single volume that can be filled with the fluid. This also has the advantage that, following the execution of the method, the cured fluid guarantees a secure connection of the various components in the desired position and orientation relative to one another. Additional connecting elements are not essential but they may be practical.

At least one of the components is preferably a joint or a fixing adapter. Here, the joint for a knee or elbow orthosis, for example, features a component main body made of the flexible material, which contains at least one partial volume. It can be connected to the various other components in the manner previously specified, so that the volume composed of the various partial volumes can subsequently filled with the curable fluid. This allows fixing adapters, joints or other functional components to be arranged in the desired position or orientation relative to one another without requiring any additional fixing elements.

The main body is preferably made from an elastic main body material. Of course, it is possible for various components that are connected to the main body to be made of different main body materials, so that different elasticities can be used. This is of course also possible if the main body is designed as a single piece.

In an especially preferred configuration, the main body is at least partially, but preferably completely, produced in an additive manufacturing process, such as a 3D printing process. To this end, for example, the body part on which the main body is to be arranged is measured using a measurement method and the resulting values are directly fed into the 3D printer. Alternatively or additionally, standard main bodies, which are available in different sizes if necessary, can be used, since they are adapted to fit the body part of the patient anyway during the execution of the method.

It is advantageous if the fluid is electrically conductive. In this way, the produced structures, i.e. the cured fluid in the volume, can be used as electrical conductor paths, for example for controlling stimulation electrodes or transmitting signals that have been obtained via myoelectric electrodes.

Preferably, different fluids are used in different regions of the volume. Of course, it is also possible that the main body features several separate volumes, each with separate supply lines, which can be filled with different fluids. In this way, a volume can be used, for instance, to guarantee the supporting function, i.e. the mechanical stability of the orthopedic device to be produced, after it has been filled with the fluid. In this case, a fluid that is particularly stable after curing can be used. The structures are often large and extend along the main force lines of the forces and loads which occur during operation of the orthopedic device. A second volume can be used, for example, in order to be filled with an electrically conductive fluid, thereby enabling electrodes or sensors to be contacted electrically. These electrodes or sensors can already be integrated in the main body or the main body features accommodation elements, such as recesses or fixing elements, on which corresponding sensors and/or electrodes can be arranged. The electric contacting of the sensors or electrodes then preferably occurs via the cured, electrically conductive fluid in this volume.

Other volumes can be filled, for instance, with a fluid that does cure but still retains a residual elasticity after curing. This is especially advantageous for a volume for which the fluid, after curing, does not or does not only have a supporting function, but in particular also performs a padding function. The different fluids in the different volumes can of course be filled with different pressures in order to meet the respective requirements.

In a preferred configuration, the cured fluid can be returned to a malleable state after curing. This can be achieved, for instance, by ensuring that the curable fluid can be returned to a liquid state or at least soften to such an extent that it is malleable again. To this end, the curable fluid preferably features at least one thermoplastic material, is produced from such a material or is composed at least partially, but preferably completely, of at least one thermoplastic material. In this way, renewed malleability can be achieved by heating the cured fluid.

This renewed malleability renders it possible to subsequently modify a main body and/or orthopedic device that has already been produced. This can be useful, for example, if the individual requirements which the patient places on the orthopedic device change during the healing process or if the orthopedic device should or must be changed or adapted for other reasons. This is the case if, for example, wearing the orthopedic device causes pain or pressure sores. This may occur with prosthesis sockets in particular and poses a serious problem that can be thus eliminated in an easy, quick and cost-effective manner.

The invention also solves the problem by way of a main body of an orthopedic device for carrying out a method described here. Such a main body thus features at least one volume with at least one supply line that can be filled with the curable fluid. It may feature several components that are or can be connected to one another. The connection between the individual components prior to filling with the curable fluid can be achieved via separate and, if necessary, removable fixing or connecting elements. Such elements can be removed following the curing of a fluid with which the volume is filled.

In the following, examples of embodiments of the present invention will be explained in more detail by way of the attached drawings: They show:

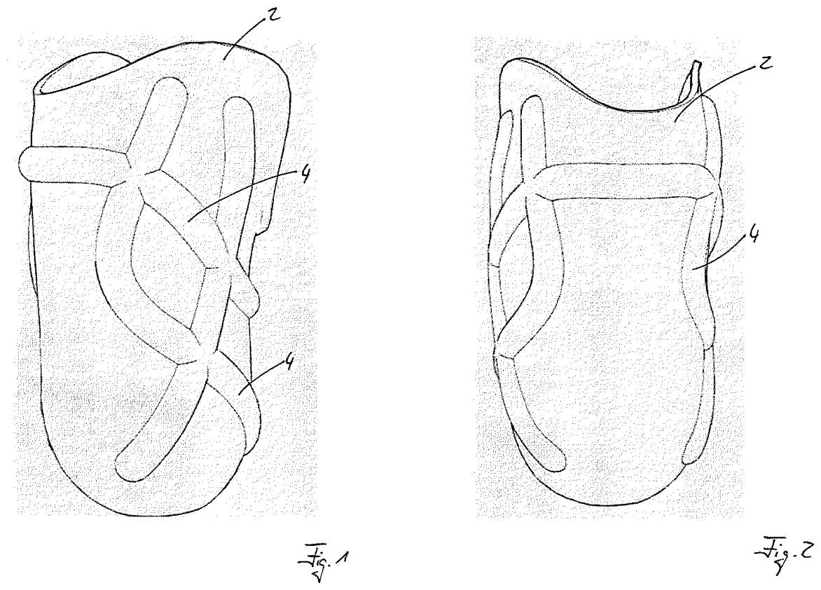

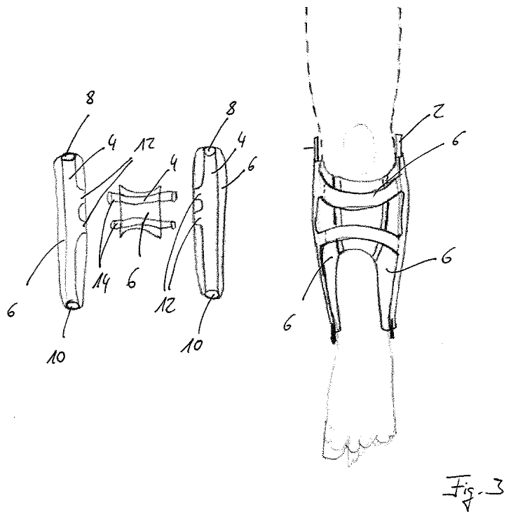

FIGS. 1 and 2—a main body of an orthopedic device according to a first example of an embodiment of the present invention,

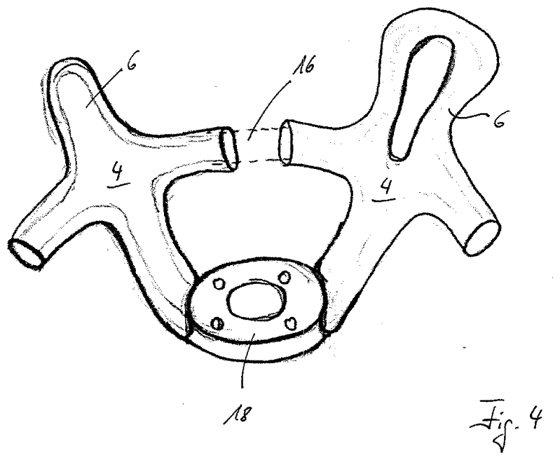

FIG. 3—the schematic representation of parts of as well as a com-plete main body according to another example of an embodiment of the present invention,

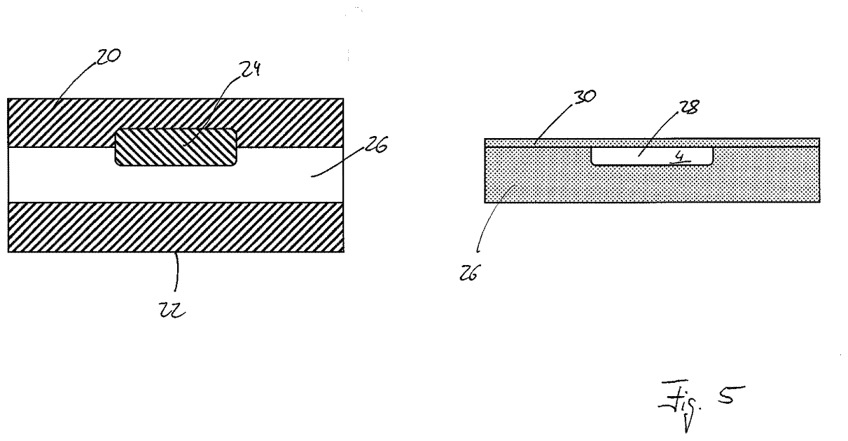

FIG. 4—a section of a main body according to a further example of an embodiment of the present invention,

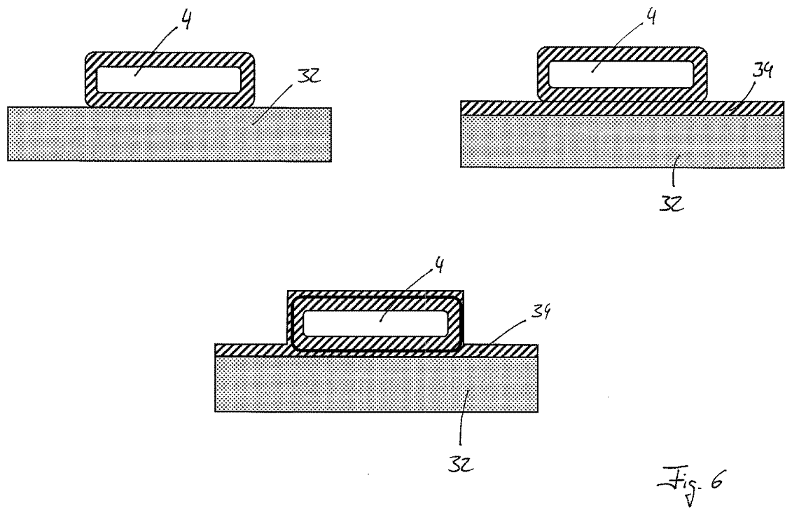

FIG. 5—the schematic representation of steps in the production of ducts,

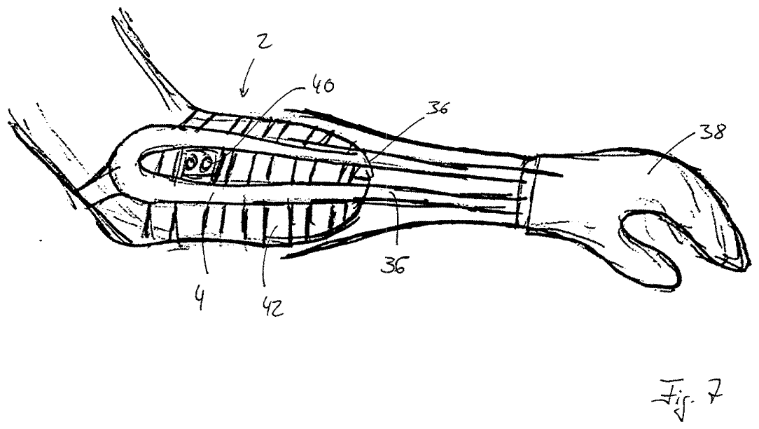

FIG. 6—various embodiments of arranged ducts according to further examples of an embodiment of the present invention, and

FIG. 7—an orthopedic device according to a further example of an embodiment of the present invention.

FIGS. 1 and 2 depict a main body 2, which in the example of an embodiment shown is a prosthesis socket. However, a liner or another component could also be used as a main body. An applied volume 4 is visible on the outside of the main body, wherein said volume is filled with a fluid in the example of an embodiment shown. It is applied to the outside of the main body 2 in such a way that there are no uncomfortable elevations on the inside of the main body 2, which comes into contact with the amputation stump.

The left-hand representation in FIG. 3 shows three components 6, which each feature a fillable volume 4. While the left and right-hand components 6 comprise an upper connection 8 and a lower connection 10, via which, for instance, the volume 4 can be filled with a fluid, their sides feature contact connections 12, into which the corresponding connections 14 of the middle component 6 can be inserted. This results in a large fillable volume 4, which can be completely or partially filled with a fluid. It is thus possible, in the manner of a construction set, to compile a volume system of a fillable volume made of different components 6, wherein said volume system is adapted to fit the respective desired properties. In the right-hand representation in FIG. 3, it is clear that the three components 6 form part of a main body 2, which in this case is part of a lower leg orthosis.

FIG. 4 shows another embodiment. The two components 6, which each feature a volume 4, can be connected to one another via a connecting adapter 16 in such a way that the two volumes 4 can form a joint fillable volume. The connecting adapter 16 is only shown via a dashed line in FIG. 4. The two components 6 are part of a main body 2, which comprises, among other things, a fixing element 18, to which additional prosthesis parts, for instance, can be arranged.

FIG. 5 illustrates a production method for the production of a corresponding volume. In the left-hand representation in FIG. 5, a part of the main body 2 is first produced, for example cast, between an upper boundary mould 20 and a lower boundary mould 22. An insert 24 is used to determine the shape of volume 4, which can be filled with the fluid in the finished main body 2. This results in a first main body element 26, which is depicted again in the right-hand representation in FIG. 5. The cavity 28 created by the insert 24 can be seen. A second main body element 30, which is connected to the first main body element 26, turns the cavity 28 into a fillable volume 4.

FIG. 6 depicts a range of embodiments, such as a produced volume 4, in the form of a duct in the example of an embodiment shown, which can be arranged on a main body material of an orthopedic device. The top-left of FIG. 6 shows the volume 4 stuck to a main body element 32. Of course, it can also be welded on or fixed in another manner. The top-right of FIG. 4 depicts a textile layer 34 arranged on the main body element 32, wherein the fillable volume 4 is arranged on said textile layer. The bottom of FIG. 6 shows the fillable volume, which is designed in the same way in all three representations; here, it is embedded in the textile layer 34 that is arranged on the main body element 32.

FIG. 7 depicts an orthopedic device in the form of a lower arm and hand prosthesis. It features the main body 2, which comprises the fillable volume 4, which is already filled with fluid in the example of an embodiment shown. It extends in two arms 36 from the elbow region to the prosthetic hand 38. An electrode arrangement 40 is positioned between the two arms 36, which is protected from mechanical influences by the arms 36 surrounding it. The main body 2 comprises circumferential reinforcement elements 42 that enable mechanical stability to be achieved.

### REFERENCE LIST

2 main body
4 volume
6 component
8 upper connection
10 lower connection
12 contact connection
14 connection
16 connecting adapter
18 fixing element
20 upper boundary mould
22 lower boundary mould
24 insert
26 first main body element
28 cavity
30 second main body element
32 main body element
34 textile layer
36 arm
38 prosthetic hand
40 electrode arrangement
42 reinforcement element

1. A method for producing an orthopedic device, comprising:
providing a main body of the orthopedic device that comprises:
at least one volume that can be filled with a fluid;
at least one supply line to this volume;
at least one of a flexible material or an elastic material in at least some sections of the main body in which there is no volume that can be filled with a fluid;
arranging the main body on a body part of a patient;
at least partially filling the volume with a curable fluid;
curing the curable fluid.

2. The method according to claim 1, wherein the fillable volume is situated at least partially in the at least one of a flexible material or an elastic material.

3. The method according to claim 1, wherein the cured fluid forms bar-shaped or finger-shaped parts, wherein a section of the at least one of a flexible material or an elastic material is situated between at least two of these parts.

4. The method according to claim 1, wherein the main body is configured to be molded to the patient's body part after the volume has been at least partially filled with the fluid.

5. The method according to claim 1, wherein the base body comprises several components, each of which features at least one partial volume that can be filled with the fluid.

6. The method according to claim 5, wherein the components are joined together to form the main body, wherein at least two are joined together to form the volume.

7. The method according to claim 5, wherein the at least one component is a joint or a fixing adapter.

8. The method according to claim 1, wherein the main body is made from an elastic main body material.

9. The method according to claim 1, wherein the main body is at least partially produced in an additive process.

10. The method according to claim 1, wherein the fluid is electrically conductive.

11. A main body of an orthopedic device for conducting a method according to claim 1.

12. A method for producing an orthopedic device, comprising:
providing a main body of the orthopedic device having at least one volume that can be filled with a fluid, at least one supply line to this volume, a plurality of sections where no fluid can be stored, and at least one of a flexible material or an elastic material in at least some sections of the main where no fluid can be stored;
arranging the main body on a body part of a patient;
at least partially filling the volume with a curable fluid;
curing the curable fluid.

13. The method according to claim 12, wherein that the at least one volume is situated at least partially in the at least one of a flexible material or an elastic material.

14. The method according to claim 12, wherein the cured fluid forms bar-shaped or finger-shaped parts, and a section of the at least one of a flexible material or an elastic material is situated between at least two of these parts.

15. The method according to claim 12, wherein the main body is configured to be molded to the patient's body part after the volume has been at least partially filled with the fluid.

16. The method according to claim 12, wherein the base body comprises several components, each of which includes at least one partial volume that can be filled with the fluid.

17. The method according to claim 16, wherein the components are joined together to form the main body, and at least two of the components are joined together to form the volume.

18. The method according to claim 16, wherein the at least one component is a joint or a fixing adapter.

19. The method according to claim 12, wherein the main body includes an elastic main body material.

20. The method according to claim 12, wherein the main body is at least partially produced in an additive manufacturing process.
